(19) United States
(12) Patent Application Publication
(10) Pub. No.: US 2019/0339184 A1
(43) Pub. Date: Nov. 7, 2019

(54) SODA ANALYSIS SYSTEM AND METHOD

(71) Applicant: David B. Barnes, Lafayette, IN (US)

(72) Inventor: David B. Barnes, Lafayette, IN (US)

(21) Appl. No.: 15/972,202

(22) Filed: May 6, 2018

(51) Int. Cl. G01N 9/14; G01N 33/14

(52) U.S. Cl. CPC G01N 9/14; G01N 33/143

(57) ABSTRACT

A system and method are disclosed for differentiating sugar-free and regular carbonated beverages. The system and method exploit the difference in specific gravity of fluid materials by observing calibrated beads immersed in fluids.

### FIELD

A system and method of differentiating diet (sugar-free) and regular (sugar sweetened) carbonated beverages via the specific gravity of a calibrated bead immersed in a sample of the beverage in a capped vial.

### BACKGROUND

Many individuals choose non-caloric (sugar-free; or also known as diet) versions of beverages rather than the standard sugar sweetened (regular) option. There are a multitude of reasons for this.

When ordering sugar-free beverages at a restaurant or similar establishment where the individual placing the order does not see the beverage or its label when the beverage is being prepared, there is always risk that the drink provided is erroneously a regular (sugar sweetened) version of the drink. There are many reasons that this mistake can occur. This risk is heightened when ordering fountain beverages where dispensing systems are complex and much of the delivery system/process is not visible. Some illustrations of the problem are presented at the website mysugr.com/sugar-free-drink-really-sugar-free-check-sugar.

Beverage manufacturers strive to make sugar-free versions indistinguishable from the regular versions. This makes the mistake difficult to recognize without some form of assistance or testing.

Mistakenly consuming a regular beverage when believing it is a sugar-free beverage can have serious consequences. In the specific case of a type 1 diabetic this error can lead to a life-threatening situation.

One means to determine if a beverage is sugar-free or regular is to use a blood glucose meter to test the beverage. The carbonated beverage may be outside the range of the blood glucose meter or the operating temperature range (43-111° F.). Additionally, the glucose test strips are an additional expense.

Another means to determine is a beverage is sugar-free or regular is to use reagent test strips for urinalysis to measure both ketones and glucose. The strips are dipped into the beverage, and if it is not sugar free, the glucose testing pad will turn darker within 30 seconds.

Specific gravity is used to distinguish regular coke and sugar-free coke in a common science demonstration. A can of regular carbonated beverage and a can of sugar-free carbonated beverage are placed in a beaker of water. The regular beverage will sink to the bottom while the sugar-free beverage will float because the actual density of the regular beverage is 1.042 g/mL and sugar-free beverage is 0.997 g/m L.

The most common method to measure specific gravity of a solution is to use a hydrometer. Hydrometers are also most commonly made of glass and are fragile. To be practical for the subject application the device should to be easily carried in a purse or pocket. The device needs to be durable and small in size.

A unique and significant challenge exists in measuring specific gravity in a carbonated liquid as bubbles spontaneously form and attach to the measurement bead confounding the test results. A method to avoid or eliminate the bubble formation is helpful to produce accurate test results.

International PCT patent publication no. WO1992005422A1, by Amer et al., discloses a device and method to measure the specific gravity of liquids. The '422 discloses a method of determining the specific gravity of a liquid comprising mixing the liquid with one or more discrete bodies or amounts of solid or liquid testing material immiscible with the liquid. The '422 utilizes bodies with predetermined specific gravities approximating or different from the specific gravity of the liquid being tested, each body being preferably visibly distinguishable, as by size, shape and/or color, from each other body with a different specific gravity. The '422 discloses observing which body or bodies float, indicating the specific gravity of the liquid being tested to be above or more than that of the floating body or bodies, and which body or bodies sink or submerge, indicating the specific gravity of the liquid being tested to be below or less than that of the sunken or submerged body or bodies. The '422 does not disclose testing to differentiate between sugar-free and regular carbonated beverages. The '422 does not disclose testing of carbonated liquids or liquids containing bubbles or dissolved gas. The '422 does not disclose a pressure tight cap or shaking a carbonated beverage in a closed container to accumulate headspace pressure. The '422 does not disclose shaking of the container leading to increased headspace pressure and elimination of bubbles in a carbonated beverage sample during testing.

U.S. Pat. No. 8,810,417 B2, by Hood et al., discloses systems that include beverage immersates with detection capability and methods of their use. A specific user utilizes components of the system in an individualized fashion, for example a person wishing to minimize their exposure to a specific substance otherwise not found objectionable by the general public may test their own beverages prior to drinking. The '417 discloses for example, a diabetic may monitor glucose in their beverages. Systems disclosed in the '417 include: at least one beverage immersate, wherein the beverage immersate includes at least one sensor configured to detect at least one analyte in a fluid within a personal use beverage container, and at least one signal transmitter configured to transmit a signal responsive to the at least one beverage immersate. The '417 discloses generating a signal in response to detection of a pressure range and a beverage container including a lid or cover, but it is silent regarding a pressure tight cap. The '417 does not explicitly disclose shaking of the container leading to increased headspace pressure and elimination of bubbles in a carbonated beverage sample during testing.

U.S. Pat. No. 3,386,289 A, by Paul D. Norcross, discloses a portable, pocket-sized apparatus for testing the specific gravity of a liquid. The apparatus comprises an elongated glass tube, one end of the glass tube is connected to a bulb that is adapted for manual manipulation to effect drawing and expulsion of liquid and air into the tube. The glass tube contains a plurality of balls of varying specific gravities that float and sink in liquids of different specific gravities when the liquid is drawn into the tube. The '289 does not disclose differentiation of sugar-free and regular carbonated beverages. The '289 does not disclose testing of carbonated liquids or liquids containing bubbles or dissolved gas. The '289 does not disclose a pressure tight cap or shaking a carbonated beverage in a closed container to accumulate headspace pressure. The '289 does not disclose shaking of the container leading to increased headspace pressure and elimination of bubbles in a carbonated beverage sample during testing.

U.S. Pat. No. 3,460,395 A, by Joseph D. Shaw, discloses a fluid measuring device. The '395 discloses an apparatus for continuously indicating the specific weights of urine specimens discharged from human patients through conventional indwelling catheters. The apparatus includes a sealed, fluid collecting vessel that has a restricted fluid inlet opening in the upper end and an air-tight connection between said opening and a flexible catheter tube. A restricted discharge opening is formed in the bottom of the vessel whereby a predetermined volume of fluid, less than the full capacity of the vessel, can be collected and retained in said vessel. A hydrometer means is freely contained in the vessel for immersion in the collected fluid and a sighting means is provided for the vessel to take direct readings on the hydrometer exteriorly of the vessel. The '395 does not disclose testing of solutions other than urine nor differentiation of sugar-free and regular carbonated beverages.

### SUMMARY

The proposed invention provides a miniature hydrometer device and method to test a beverage to determine whether it is a sugar-free or regular version of a served beverage.

The soda analysis system and method of use exploits the difference in specific gravity to distinguish between sugar-free and regular versions of carbonated beverages and corrects for inaccuracies caused by carbonated bubbles.

In one aspect of the invention, a soda analysis system for determining if a carbonated beverage is sugar-free or regular is disclosed. The soda analysis system is comprised of a vial with a pressure tight cap and a bead with a specific gravity between approximately 0.997 and approximately 1.042 g/m L.

In a further aspect of the invention, a method is disclosed to determine if a carbonated beverage is sugar-free or regular via a soda analysis system comprised of a vial with a pressure tight cap and a bead with a specific gravity between approximately 0.997 and approximately 1.042 g/mL.

In additional aspects of the invention, the bead may comprise Nylon 12 or a combination of other materials that provides the required specific gravity.

### BRIEF DESCRIPTION OF THE DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 is an exploded view of the components of the soda analysis system.

FIG. 2 illustrates a bead of FIG. 1 floating in a capped vial of a sugar-free carbonated beverage.

FIG. 3 illustrates a bead of FIG. 1 sunk in a capped vial of a regular carbonated beverage.

FIG. 4 is a photograph of the soda analysis system.

FIG. 5 is a photograph of the soda analysis system containing a test solution of sugar-free carbonated beverage with the vial cap off and without agitation.

FIG. 6 is a photograph of the soda analysis system containing a test solution of regular carbonated beverage with the vial cap off and without agitation.

FIG. 7 is a photograph of the soda analysis system containing a test solution of sugar-free carbonated beverage after agitation.

FIG. 8 is a photograph of the soda analysis system containing a test solution of regular carbonated beverage after agitation.

Corresponding reference characters indicate corresponding parts throughout the several views. Although the drawings represent embodiments of the present disclosure, the drawings are not necessarily drawn to scale and certain features may be exaggerated in order to better illustrate and explain the present disclosure.

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

There are several ranges of specific gravity which may work between approximately 0.997 and approximately 1.042 g/mL, such as between approximately 1.005 and approximately 1.035, between approximately 1.01 and approximately 1.03, between approximately 1.015 and approximately 1.025, and between approximately 1.0175 and approximately 1.0225. An exemplary testing bead would have a specific gravity at the midpoint of the sugar-free to sugar sweetened range which is about 1.02. Use of a standard, off-the-shelf material which may vary slightly from this about 1.02 value may be an economical and acceptable trade-off.

The spherical bead is comprised of a blend of two common plastics in proportions needed to offer the desired composite density. Testing of a layered pellet manufactured of varying parts polypropylene (PP) and acrylonitrile butadiene styrene was conducted with gradual shaving off of the PP side of the pellet until the proper combined density was obtained. The result indicated that minimal PP was needed to reach the ideal density.

A review of standard materials by Stelray Plastic Products (Anisonia, Conn.; www.stelray.com/wp-content/uploads/2018/03/Reference-Tables-Density-of-plastic.pdf) indicated that beads could be produced that would fall within the precise specific gravity (SG) range required to provide correct identification of the tested solution with high accuracy such as Nylon 12, and beads made of a 50/50 mixture of various other materials including PP, acrylonitrile butadiene styrene, Polycarbonate, PVC and Nylon 6.

Due to the inherent variation in any mass manufacturing process, beads purchased in bulk should be precisely calibrated to ensure test accuracy by use of a two-step process. First the beads are placed in a first test fluid bath of SG=1.00 g/mL. Any bead that does not sink is removed and discarded. Secondly the remaining beads are placed in a second test fluid bath of SG=1.02 g/mL. Any bead which does not float in the second test fluid bath is removed and discarded. The remaining beads are confirmed valid for the soda analysis system. In this way, the ultra-high precision required for test beads may be obtained at a relatively low cost, yet with full confidence of test accuracy.

The two test fluid baths can be calibrated using a scientific hydrometer such as can be purchased from Grainger (www.grainger.com/product/VEE-GEE-Hydrometer-Replacement-48ME52). Distilled water may be a very acceptable solution for the first calibration bath (SG=1.00). Sugar can be added to distilled water incrementally until the desired specific gravity is reached for the 2nd calibration bath. A carbonated solution may also be used for both test fluid baths.

If beads made from Nylon 12 were used which has a nominal specific gravity of 1.02, very few beads would be rejected as a result of the calibration testing described in paragraph [0029]. If a material was chosen more towards the extents of any exemplary range, such as SG=1.006, more of the beads would be rejected in the calibration test leading to increased scrap expense. After the calibration test, all calibrated testing beads would be acceptable for use regardless of their material composition.

The proposed invention combines the calibrated testing bead 105, a vial 101 and a suitable pressure tight cap 103. The capped vial will ideally be small, portable, durable, of a transparent material and able to withstand internal pressures greater than those produced by carbonated beverages when agitated.

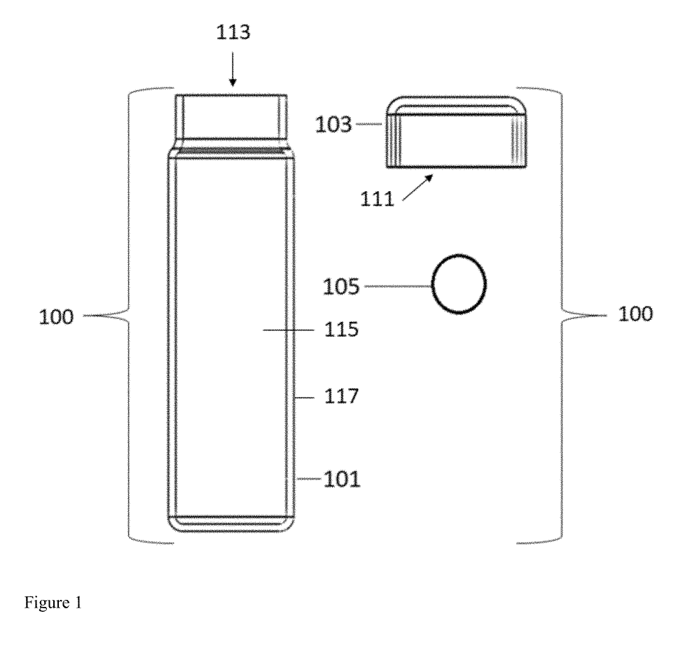

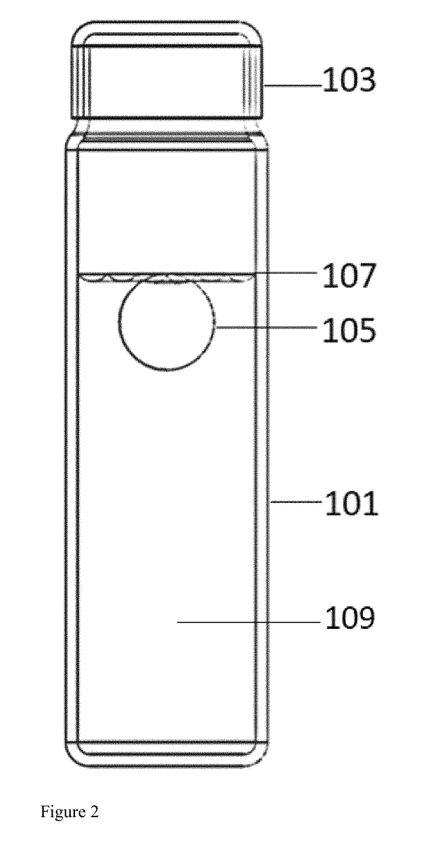

As illustrated in FIG. 1 the soda analysis system 100 comprises a threaded and transparent vial 101, a pressure tight screw on cap 103 including seal 111, and a calibrated bead 105. Bead 105 benefits from being as large as possible while still fitting through vial opening 113. A large bead 105 leaves a few millimeters of clearance once the bead 105 is down into the main body 115 of the vial 101. Too much clearance between bead 105 around vial opening 113 or vial wall 117 may make it difficult to “see” the bead in a dark colored beverage 109, 110 (FIGS. 2, 3, and 5-8). Too little clearance between bead 105 around vial opening 113 or vial wall 117 may cause interaction between the bead 105 and vial wall 117 which may reduce test robustness. Current testing has shown 4-5 mm total clearance between the vial wall 117 and bead 105 when fully inserted into the vial 101 to perform well. Test performance was not found to be highly sensitive to the clearance.

The soda analysis system 100 is used in a method to determine if a carbonated beverage is sugar-free or regular. The method comprises the steps of placing the bead 105 in the vial 101, adding a sample of the solution to be tested in an amount sufficient to fill the vial to an indicated level 107, tightening the cap 103 to the vial 101 sufficient to ensure the cap is pressure tight and shaking the capped vial of solution, ensuring that the agitation dislodged and disseminated any bubbles that may have attached to the bead 105, and then determining the position of the bead in the solution.

As illustrated in FIG. 2, the bead 105 is floating which confirms, the solution 109 to be “regular” or “sugar containing”.

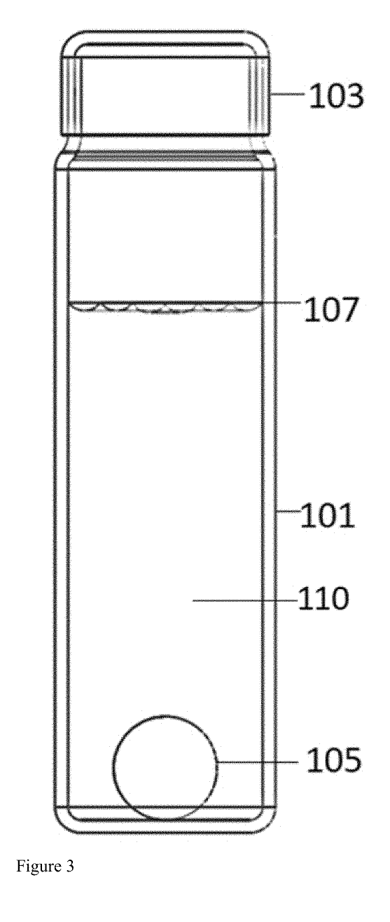

As illustrated in FIG. 3, the bead 105 sinks which confirms the solution 110 to be “sugar-free”.

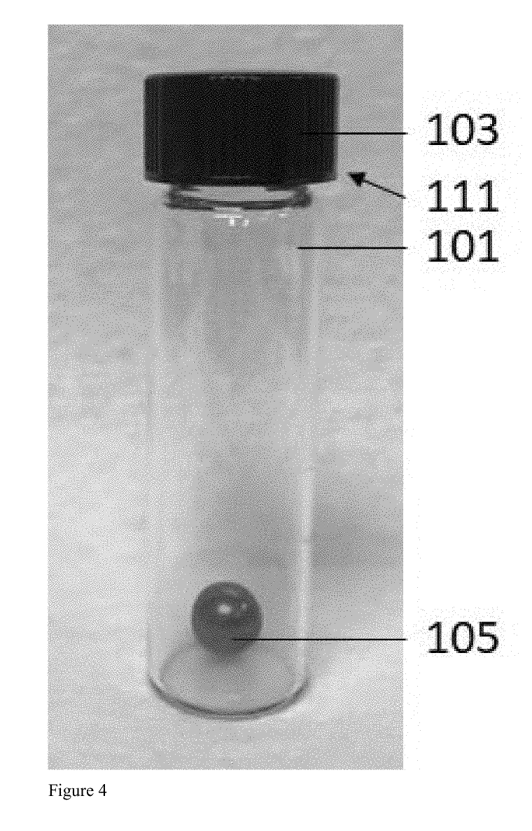

The soda analysis system 100, as shown in FIG. 4, comprises a threaded and transparent vial 101, a pressure tight screw on cap 103 including seal 111, and a calibrated bead 105.

There are means to address a specific problem of bubbles adhering to the bead, which may compromise test accuracy. If the vial cap is sealed and the carbonated beverage is agitated, head pressure within the vial will serve to suppress the formation of carbonation bubbles on the bead. Without shaking, the closed vial will not accumulate head pressure fast enough for the bead to be unaffected by the bubbles and give a reliable reading of the specific gravity of the beverage. Alternatively, a sealed vial without agitation may eventually exhaust the formation and attachment of bubbles to the bead, but it is time consuming to wait for the bubbles to dissipate. As another alternative, it is envisioned that some type of coating on the bead might reduce adhesion of bubbles to the bead.

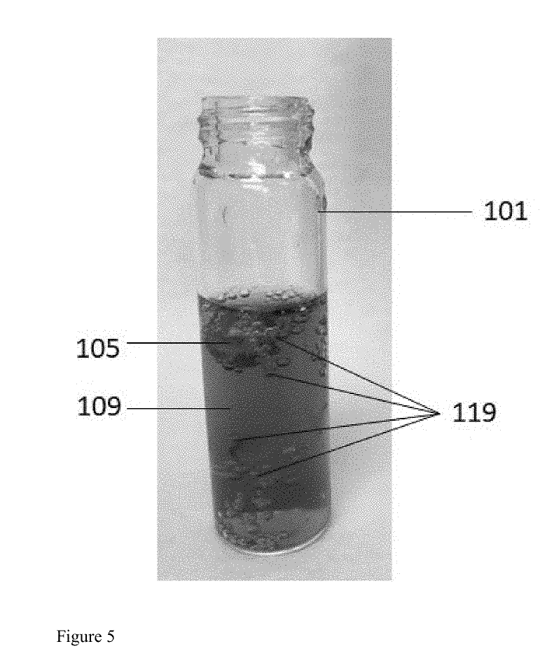

FIG. 5 shows vial 101 filled with a test solution of sugar-free carbonated beverage 109 with the soda analysis system not capped and without agitation. Without head pressure, bubbles 119 form and cause bead 105 to float and interfere with the analysis.

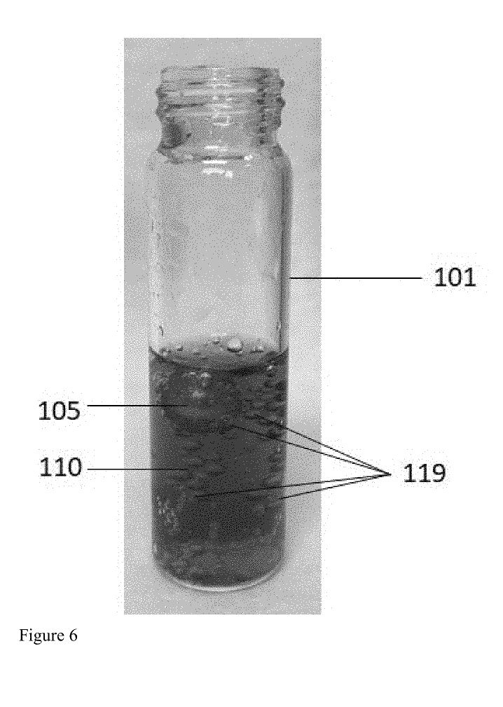

FIG. 6 shows vial 101 filled with a test solution of regular carbonated beverage 110 with the soda analysis system not capped and without agitation. Without head pressure, bubbles 119 form and cause bead 105 to float and interfere with the analysis.

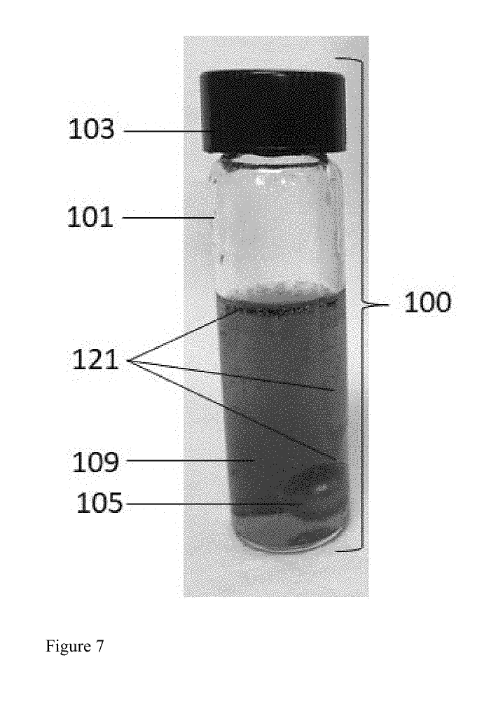

FIG. 7 shows the soda analysis system 100 filled with a test solution of sugar-free carbonated beverage 109 after agitation under sufficient head pressure to suppress the formation of bubbles on sunken bead 105 that may interfere with analysis. Some bubbles 121 are present, but they do not interfere.

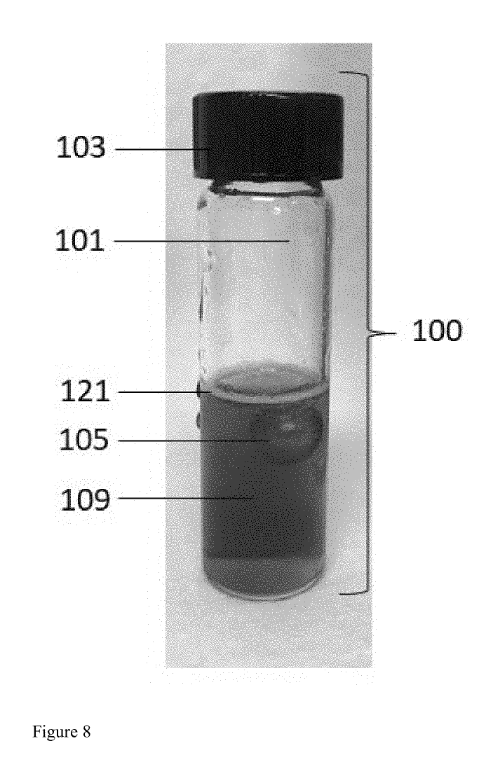

FIG. 8 shows the soda analysis system 100 filled with a test solution of regular carbonated beverage 110 after agitation under sufficient head pressure to suppress the formation of bubbles on floating bead 105 that may interfere with the analysis. However, some bubbles 121 are present.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

What is claimed is:

1. A system to determine if a carbonated beverage is sugar-free or regular comprising:
a vial, a pressure tight cap, and a bead, wherein the bead has a specific gravity between approximately 0.997 and approximately 1.042 g/mL,
wherein the bead sinking indicates a sugar-free carbonated beverage, and
wherein the bead floating indicates a regular carbonated beverage.

2. The system of claim 1 wherein the bead composition is selected from the group consisting of Nylon 12, a combination of polypropylene and acrylonitrile butadiene styrene, and a mixture of polypropylene and Nylon 6.

3. The system of claim 3 wherein the bead composition is Nylon 12.

4. The device system of claim 3 wherein the bead composition is a combination of polypropylene and acrylonitrile butadiene styrene.

5. The system of claim 3 wherein the bead composition is a mixture of polypropylene and Nylon 6.

6. The system of claim 1 wherein the bead has a specific gravity selected from the group consisting of the range 1.00 to 1.04 g/mL., 1.00 to 1.03 g/mL, and 1.00 to 1.02 g/mL.

7. The system of claim 6 wherein the bead has a specific gravity within the range 1.00 g/mL to 1.04 g/mL.

8. The system of claim 6 wherein the bead has a specific gravity within the range 1.00 to 1.03 g/mL.

9. The system of claim 6 wherein the bead has a specific gravity within the range 1.00 to 1.02 g/mL

10. A method to determine if a carbonated beverage is sugar-free or regular comprising the steps of:
adding the carbonated beverage to a vial containing a bead,
closing a pressure tight cap on the vial,
increasing head pressure within the vial and
determining the location of the bead in the vial.

11. The method of claim 10 further comprising the step of:
shaking the capped vial including the carbonated beverage after closing the cap and before determining the location of the bead.

12. The method of claim 11 wherein shaking results in increased head pressure in the vial.

13. The method of claim 10 wherein the bead floating indicates a regular carbonated beverage or the bead sinking indicates a sugar-free carbonated beverage.

14. The method of claim 10 wherein the step of increasing head pressure prevents bubbles from forming and adhering to the bead.

15. The method of claim 10 further comprising the step of swirling or tapping the vial to remove bubbles from the bead.

16. A method of testing the specific gravity of a bead comprising the steps of:
providing a first test fluid bath calibrated to a specific gravity of 1.00 g/mL,
placing the bead in the first test fluid bath,
removing the bead from further testing if the bead does not sink,
providing a second test fluid bath calibrated to a specific gravity of 1.02 g/mL or higher,
placing the bead in the second test fluid bath,
removing the bead from further testing if the bead does not float.

17. The method of claim 16 wherein the second bath has a specific gravity within the range of 1.02 g/mL to 1.04 g/mL.

18. The method of claim 16 wherein the second bath has a specific gravity selected from the group consisting of 1.02 g/mL, 1.03 g/mL, and 1.04 g/mL.
